(19) United States
(12) Patent Application Publication
(10) Pub. No.: US 2011/0202140 A1
(43) Pub. Date: Aug. 18, 2011

(54) LOAD BEARING IMPLANTS WITH ENGINEERED GRADIENT STIFFNESS AND ASSOCIATED SYSTEMS AND METHODS

(75) Inventors: Alexander C. Turner, Santa Fe, NM (US); Rajendra Kumar Bordia, Seattle, WA (US)

(73) Assignee: UNIVERSITY OF WASHINGTON, Seattle, WA (US)

(21) Appl. No.: 13/026,999

(22) Filed: Feb. 14, 2011

Related U.S. Application Data
(60) Provisional application No. 61/303,846, filed on Feb. 12, 2010.
(60) Provisional application No. 61/305,471, filed on Feb. 17, 2010.

(51) Int. Cl. A61F 2/32; A61F 2/30

(52) U.S. Cl. 623/22.4; 623/18.11

(57) ABSTRACT

Implants are made of materials having asymmetric modulus gradients. For example, an implant, such as a hip implant, is made of a material having a stiffness gradient between a proximal portion near a hip joint and a distal portion extending downward into the marrow of the femur. Among other benefits, the asymmetric modulus gradient mitigates problems associated with stress shielding and does not excessively wear or deteriorate the proximal portion of the implant.

### CROSS-REFERENCE TO RELATED APPLICATIONS

This application claims priority to pending U.S. Provisional Application No. 61/303,846, filed Feb. 12, 2010, and pending U.S. Provisional Application No. 61/305,471, filed Feb. 17, 2010, both of which are incorporated herein by reference in their entireties.

### TECHNICAL FIELD

The present disclosure is directed generally to load bearing implants with engineered gradient stiffness and associated systems and methods.

### BACKGROUND

Bone and joint implants have improved the lives of many people who suffer from injury or disease by restoring mobility and even athleticism to patients. As the medical science advances and matures, however, problems have arisen with conventional implants. Natural bones are rigid but flexible. As a person or animal moves about, their bones experience natural mechanical stresses due to muscular loading and impacts that cause the bones to maintain a healthy density and even remodel. In the absence of natural mechanical stresses, bones tend to lose density. This phenomenon, known as Wolffs law, is a well-known scientific principle. Conventional implants are generally more rigid than bone, and are made of a solid material such as commercially pure titanium (“CPTi”) that can prevent these natural stresses from reaching the bone. This is known as stress shielding.

FIG. 1, for example, is a schematic view of an implant 10 made of a fully dense material according to the prior art. Fully dense CPTi is approximately 1% porous or less, and has a high stiffness of approximately 110 GPa. Because the implant 10 is made of uniformly dense material, the modulus of elasticity or stiffness of the implant 10 is generally uniform throughout the implant 10. Thus, this implant 10 is much more rigid than the surrounding bone 14 and can cause stress shielding in the bone 14.

Another problem with conventional implants (such as the implant 10 of FIG. 1) is deterioration of the implant over time due to debris build-up and other factors. In an attempt to solve the stress shielding problem, some conventional designs include implants with low modulus of elasticity so that the implant will flex more like a natural bone and allow the bone to experience normal stress. Accordingly, there is a need in the art for an improved implant that overcomes these issues.

### BRIEF DESCRIPTION OF THE DRAWINGS

FIG. 1 is a schematic view of a conventional bone implant made of fully dense CPTi according to the prior art.

FIG. 2A is a schematic view of a bone implant configured in accordance with an embodiment of the present disclosure.

FIG. 2B is a schematic view of the bone implant of FIG. 2A positioned within a human femur in accordance with an embodiment of the present disclosure.

FIG. 3A is a schematic view of a bone implant having a radial modulus gradient configured in accordance with an embodiment of the present disclosure.

FIG. 3B is a schematic, cross-sectional view of the bone implant of FIG. 3A taken along line 3B-3B in FIG. 3A.

FIG. 3C is a schematic, cross-sectional view of a bone implant configured in accordance with another embodiment of the present disclosure.

FIG. 4 is a schematic view of a bone implant having an engineered modulus gradient configured in accordance with still another embodiment of the present disclosure.

FIG. 5 is a schematic view of a bone implant having an engineered modulus gradient configured in accordance with yet another embodiment of the present disclosure.

FIG. 6 is a schematic view of a bone implant having a uniform porosity of approximately 73% porosity configured in accordance with an embodiment of the present disclosure.

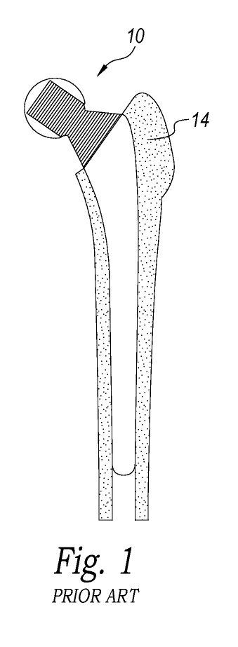

### DETAILED DESCRIPTION

The present disclosure describes load bearing implants with engineered gradient stiffness and associated systems and methods. Several embodiments of the load bearing implants described herein, for example, are directed to implants having optimized stiffness gradients and methods for designing the stiffness gradients in such implants. In one embodiment, for example, stiffness gradients for implants (e.g., hip stem implants) can be engineered using simulations (e.g., finite element analysis) to minimize bone loss due to stress shielding and also to maintain the shear stress at the bone/implant interface to be below a desired threshold value.

Mechanical properties of load bearing implants should not adversely affect the biological function and processes of surrounding anatomical structures. Specifically, implants should not adversely affect the surrounding bone (in case of joint implant) and should not compromise the bone healing (in case of implants for bone defects). As noted previously, one problem with many conventional implants is that high stiffness implant materials and configurations prevent bones from receiving normal levels of mechanical stimulation. This often results in bone loss around the implant, which can lead to pain, difficulty in revision surgery, and possible implant failure.

In contrast with conventional implants, the load bearing implants disclosed herein have non-uniform distribution of stiffness within the individual implants. This is expected to significantly reduce stress shielding while maintaining low levels of interface stress. Among other benefits, the implants and associated techniques for forming such implants disclosed herein are further expected to (a) extend the life of the implants and reduce the need for revision surgeries, (b) reduce long term pain associated with implants due to stress concentration, and (c) provide a more physiologically compatible substrate for large bone defects (e.g., plates, screws, and substrates for bone growth).

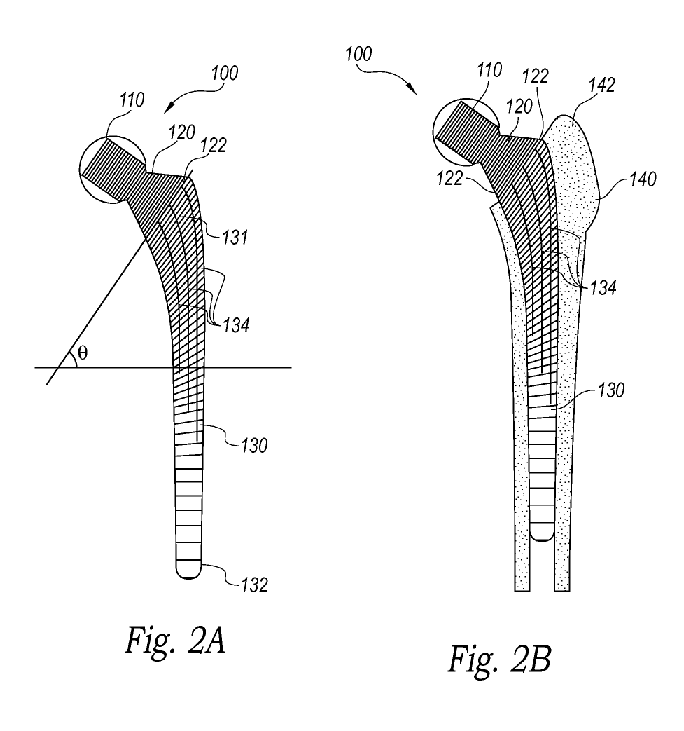

Specific details of several embodiments of the technology are described below with reference to FIGS. 2A-6. Other details describing well-known structures and systems often associated with implants have not been set forth in the following disclosure to avoid unnecessarily obscuring the description of the various embodiments of the technology. Many of the details, dimensions, angles, and other features shown in the Figures are merely illustrative of particular embodiments of the technology. Accordingly, other embodiments can have other details, dimensions, angles, and features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 2A-6.

FIG. 2A is a schematic view of a load bearing bone (i.e., hip) implant 100 configured in accordance with an embodiment of the present disclosure. While the hip implant 100 is used to describe various aspects of the present technology in this disclosure with reference to FIGS. 2A-6, it will be appreciated that the discussion herein is equally applicable to replacement implants for other body parts, such as shoulder, knee, and ankle implants, as well as a variety of other load bearing implants. Various aspects of the disclosure may also be used with substrates for large bone defects (e.g., due to injury, trauma, or disease). In still further embodiments, the implants described herein may be used for animal subjects.

The implant 100 of FIG. 2A includes a ball portion 110, a neck portion 120, and a stem portion 130. The ball portion 110 comprises a generally spherical ball configured to engage a pelvis bone of the patient (not shown) in a ball-and-socket joint (not shown). The neck portion 120 is a narrow region between the ball portion 110 and the stem portion 130. In the illustrated embodiment, the neck portion 120 is narrower at the ball portion 110 than at the stem portion 130. In other embodiments, however, the neck portion 120 may have other arrangements. The neck portion 120 interfaces with the stem portion 130 at an interface region 122 that is angled from a horizontal plane by an angle θ. The stem portion 130 comprises an elongated member extending from the neck portion 120 slightly laterally outwardly and downwardly from the neck portion 120. The stem 130 has a proximal portion 131 at the interface region 122, and a distal portion 132 at an extreme end of the stem 130. In the illustrated embodiment, the ball portion 110, the neck portion 120, and the stem portion 130 are integral components composed of the same material. For example, in some embodiments, the implant 100 can be made of commercially pure titanium (“CPTi”), titanium aluminum vanadium (“Ti₆Al₄V”), or another suitable material. In other embodiments, however, the components of the implant 100 are not all composed of the same material.

FIG. 2B is a schematic view of the implant 100 of FIG. 2A with the implant 100 inserted into the interior region of a human femur 140 in accordance with an embodiment of the present disclosure. The stem portion 130, for example, is configured to be inserted into a central or marrow region of the femur 140. The stem portion 130 can extend completely or approximately completely into the femur 140, with a small portion of the stem portion 130 protruding from the femur 140. The greater trochanter 142 of the femur 140 can extend above the interface region 122 between the neck portion 120 and the stem portion 130.

In some embodiments, the implant 100 has a varying modulus of elasticity as a function of a spatial parameter of the implant 100. The implant 100, for example, can have a modulus gradient, with the modulus of elasticity of any given point defined at least in part by a dimensional parameter of that point. For example, the implant 100 has an axial modulus gradient and the modulus is higher at the interface region 122 and decreases as a function of distance from the interface region 122, such as along gradient lines 134. The gradient can be expressed parametrically with a distance from the interface region 122 or from another reference point as the parameter by which the modulus is varied. In some embodiments, the gradient lines 134 are approximately equally spaced and mimic the profile of the implant 130. In some embodiments, the modulus at a proximal portion 131 is anywhere between 110 and 9.9 GPa (e.g. the same modulus as fully dense CPTi and 70% porous CPTi, respectively), and the modulus at a distal portion 132 vary from the values set forth above. In other embodiments, however, the modulus values at the proximal portion 131 and/or distal portion 132 can be different.

In accordance with the present disclosure, there are many ways by which the modulus of elasticity of bone implants can be varied at different positions throughout the implants. One such technique, for example, is varying the porosity of the implants. The stiffness of an implant is inversely related to the porosity level. For example, implants having a low porosity (i.e. a more dense material) have a relatively high modulus of elasticity or stiffness. Likewise, implants with greater porosity have a relatively lower modulus of elasticity or stiffness. The implant 100 of FIGS. 2A and 2B can have varying porosity levels of anywhere between 0% porous (fully dense) and 90% porous. At 0% porosity, the implant 100 has a modulus of elasticity of approximately 110 GPa; at 90% porosity, the modulus of the implant 100 is approximately 1.1 GPa. In some embodiments, the modulus of elasticity varies linearly between 110 GPa and 1.1 GPa as the porosity varies between 0% and 90% porosity. In other embodiments, however, the porosity may be different.

In selected embodiments, the mechanism by which varying porosity levels are formed in the implant 100 include the Electron Beam Melting (“EBM”) method, the Laser Engineered Net Shaping (“LENS”®) method, or another suitable method. These methods are described in more detail in U.S. Provisional Application Nos. 61/303,846 and 61/305,471. As provided above, both of these applications are incorporated herein by reference in their entireties.

Conventional implants having a low porosity and high modulus may be prone to stress shielding. The inventors in the present application have discovered that a desirable porosity that minimizes the potential difficulties can vary as a function of dimensional and material parameters of the implant. For example, the inventors in the present application have discovered that implants with high stiffness proximally and decreasing stiffness distally (such as the implant 100 of FIGS. 2A and 2B) provide significant improvements in bone stimulation (measured in terms of strain energy density) relative to a conventional fully-dense titanium (Ti) implant and an optimized uniformly porous implant.

Another feature of the implant 100 is that the implant 100 has been numerically designed and optimized (e.g., using finite element analysis) to determine a desirable porosity and gradient configuration for a given implant size, material, and position in the body. The inventors have further discovered that such engineered implants outperform fully dense or uniform porosity implants in bone adaptation studies that simulate bone loss following implantation. Implants having the modulus gradients discussed herein allow the bone to experience natural mechanical stresses that stimulate healthy bones.

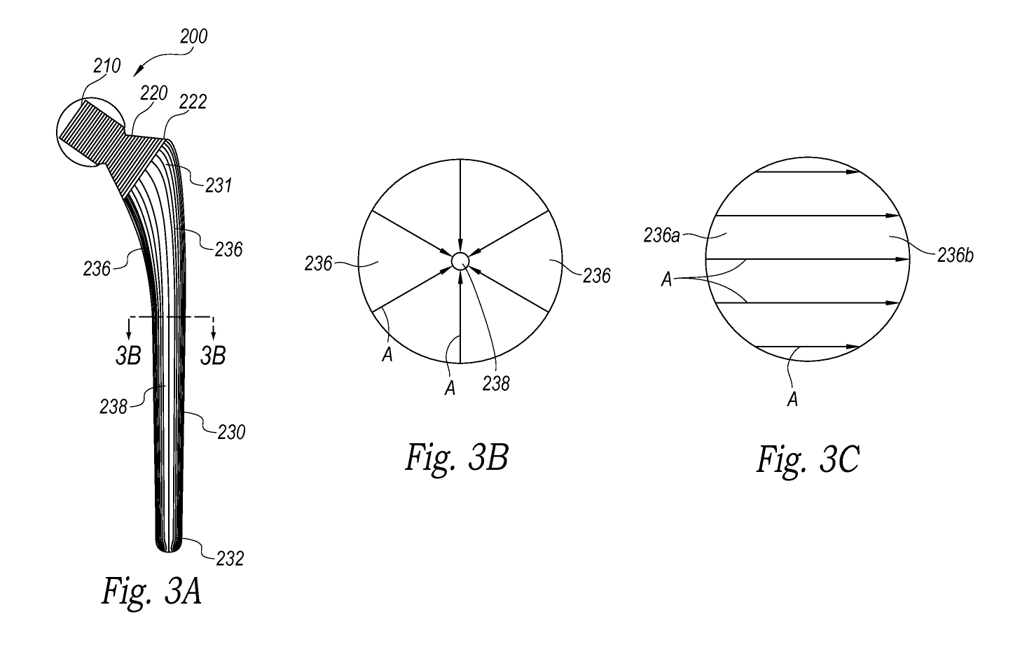

FIG. 3A is a schematic view of a load bearing implant 200 configured in accordance with another embodiment of the disclosure. The implant 200 can have a number of features generally similar to the implant 100 described above with reference to FIGS. 2A and 2B. For example, the implant 200 includes a ball portion 210, a neck portion 220, and a stem portion 230. The stem portion 230 has a proximal portion 231 at an interface region 222, and a distal portion 232 at an extreme end of the stem 230. The implant 200 differs from the implant 100 of FIGS. 2A and 2B in that the implant 200 comprises a radial modulus gradient rather than an axial modulus gradient. The radial modulus gradient, for example, is a generally concentric radial modulus in which peripheral regions 236 of the implant 200 are more rigid (higher modulus of elasticity) than an inner or center region 238 of the implant 200. In other embodiments, however, this arrangement may be reversed and the center region 238 may be more rigid than the peripheral regions 236.

FIG. 3B, for example, is a schematic, cross-sectional view of the implant 200 taken along line 3B-3B in FIG. 3A. As best seen in FIG. 3B, the stem 230 of the implant 200 comprises a generally concentric radial modulus gradient (as shown by the arrows A). FIG. 3C is a schematic, cross-sectional view of the implant 200 illustrating still another embodiment in which the modulus gradient comprises a unilateral gradient (as shown by the arrows A), with a laterally interior side 236a having a higher (or lower) stiffness than a laterally exterior region 236b. In other embodiments, the unilateral gradient is oriented in other directions, such as front to back, exterior to interior, or any other suitable orientation. In still other embodiments, the radial modulus gradient of the implant 200 may have other arrangements.

FIG. 4 is a schematic view of a portion of a load bearing implant 300 with an engineered modulus gradient configured in accordance with still another embodiment of the present disclosure. The implant 300 can have a number of features generally similar to the implant 100 described above with reference to FIGS. 2A and 2B. For example, the implant 300 includes a ball portion 310, a neck portion 320, and a stem portion 330. The implant 300 also includes an interface region 222 between the stem portion 330 and the neck portion 320.

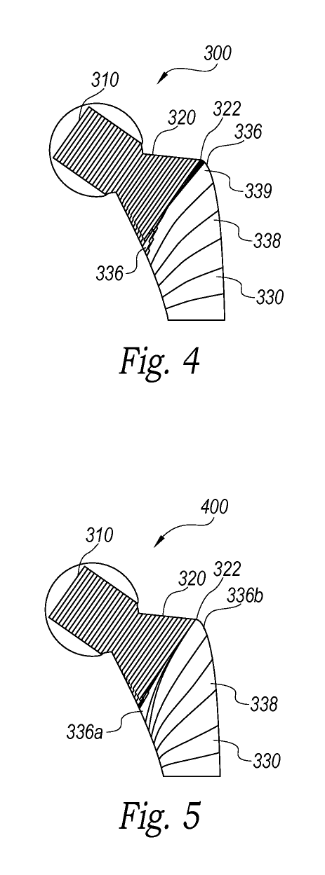

The implant 300 can have a localized modulus gradient along the interface region 322, where external portions 336 of the stem portion 330 are more rigid than interior portions 338 of the stem portion 330. More specifically, at the interface region 322, the stem portion 330 can have a modulus at least generally equal to the modulus of the neck region 320. The modulus gradually decreases as a function of distance from the interface region 322, similar to the arrangement described above with respect to FIGS. 2A and 2B. In this embodiment, however, as shown by the shading in FIG. 4, the gradient at an external portion 336 of the stem portion 330 decreases more gradually than at an interior region 338. FIG. 5 is a schematic view of portion of the load bearing implant 300 illustrating a related embodiment in which the engineered gradient is unilateral or asymmetric in the sense that the engineered gradient on a first side 336a of the stem portion 330 (as shown by the shading in FIG. 5) decreases more gradually than the gradient at the center region 338, and decreases still more gradually at a second side 336b of the stem portion 330 opposite the first side 336a.

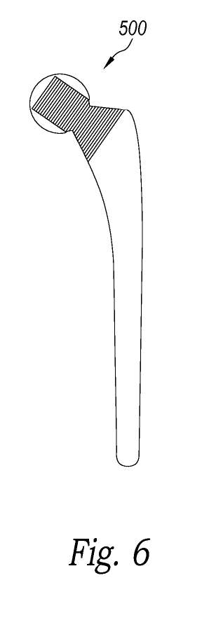

FIG. 6 is a schematic view of an implant 500 configured in accordance with a particular embodiment of the present disclosure in which the implant 500 is made of a uniformly porous material. In particular, to meet these design considerations, the implant 500 in the illustrated embodiment is composed of CPTi and has a generally uniform porosity of approximately 73%. The implant 500 has a modulus of elasticity of approximately 30.5 GPa. The relationship between porosity and modulus is a function of the material of the implant. The inventors in the present application have discovered that this particular configuration can effectively reduce stress shielding and promote healthy bone maintenance.

In selected embodiments, the implants 200, 300, 400, and 500 can have compound gradients that combine any two or more of the modulus gradients described herein. For example, an implant configured in accordance with embodiments of this disclosure may have an axial and a radial modulus gradient, an axial and an engineered gradient, or any other suitable combination of the gradients mentioned herein. In some embodiments, the gradients are non-linear gradients. The ranges of modulus of elasticity given for the embodiments described above are not limiting, and are merely used to illustrate certain features of the disclosed technology.

From the foregoing it will be appreciated that, although specific embodiments of the technology have been described herein for purposes of illustration, various modifications may be made without deviating from the spirit and scope of the technology. For example, the modulus of elasticity of the implants can be varied using a variety of techniques, including by varying the porosity of the implants. Further, certain aspects of the new technology described in the context of particular embodiments may be combined or eliminated in other embodiments. For example, in the embodiments illustrated above, various combinations of modulus gradients may be combined into a single implant. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A hip implant, comprising:
a ball portion configured to engage a pelvis bone; and
a stem portion configured to engage an interior portion of a femur bone, wherein the stem portion comprises a proximate portion attached to the ball portion and a distal portion extending from the proximate portion,
wherein the stem portion has a modulus gradient based at least in part upon varying levels of porosity from the proximate portion to the distal portion.

2. The hip implant of claim 1 wherein a chemical composition is independent of the modulus of the stem portion.

3. The hip implant of claim 1 wherein the stem portion comprises a radially interior side and a radially exterior side, and wherein the stem portion has a modulus gradient from the radially interior side to the radially exterior side.

4. The hip implant of claim 3 wherein the stem portion has a modulus gradient based at least in part upon varying levels of porosity from the radially interior side to the radially exterior side.

5. The hip implant of claim 3 wherein the modulus gradient is stiffer at the radially interior side than at the radially exterior side.

6. The hip implant of claim 1, further comprising an interface between the ball portion and the stem portion, and wherein the implant is configured to engage the femur bone with the interface just outside the interior portion of the femur bone.

7. The hip implant of claim 1 wherein the modulus gradient is stiffer at the proximate portion than at the distal portion.

8. The hip implant of claim 1 wherein the stem portion comprises a porous material of approximately 73% porosity.

9. The hip implant of claim 1 wherein the stem portion comprises at least one of commercially pure titanium (“CPTi”), and titanium aluminum vanadium (“Ti6Al4V”).

10. A bone implant, comprising:
a proximal portion;
a distal portion;
a laterally interior portion; and
a laterally exterior portion,
wherein the bone implant has an asymmetric stiffness gradient based at least in part upon a varying porosity.

11. The bone implant of claim 10 wherein the stiffness gradient is between 110 GPa and 1.1 GPa.

12. The bone implant of claim 10 wherein the varying porosity is between approximately 0% porous and approximately 90% porous.

13. The bone implant of claim 10 wherein the stiffness gradient comprises an axial gradient between the proximal portion and the distal portion, and wherein the bone implant is stiffer at the proximal portion than at the distal portion.

14. The bone implant of claim 10 wherein the stiffness gradient comprises a radial gradient between the laterally interior portion and the laterally exterior portion.

15. The bone implant of claim 10 wherein the stiffness gradient comprises an axial gradient between the proximal portion and the distal portion, and wherein the stiffness gradient further comprises a radial gradient between the laterally interior portion and the laterally exterior portion.

16. The bone implant of claim 10, further comprising an anchor portion of at least generally uniform stiffness attached to the proximal portion.

17. The bone implant of claim 10 wherein the stiffness gradient comprises a compound gradient extending between the proximate portion and the distal portion; and
between the laterally interior portion and the laterally exterior portion.

18. The bone implant of claim 17 wherein the bone implant is made using at least one of Laser Engineered Net Shaping (LENS®) and Electron Beam Melting (EBM).

19. A bone implant, comprising:
a bone marrow engaging portion configured to be inserted within an interior region of a bone; and
an exposed portion attached to the bone marrow engaging portion, wherein the exposed portion is configured to protrude from the bone, and wherein the bone marrow engaging portion has an asymmetric stiffness gradient based at least in part upon varying porosity levels in the bone implant.

20. The bone implant of claim 19 wherein the stiffness gradient comprises at least one of an axial gradient extending from the exposed portion through the bone marrow engaging portion and a radial gradient extending from a first side of the bone marrow engaging portion and a second side of the bone marrow engaging portion.

21. The bone implant of claim 19 wherein the stiffness gradient comprises a range of stiffness between approximately 110 GPa and 1.1 GPa.
